SEQUENCE LISTING (14 sequences)

SEQ ID NO: 1 - protein, 295 aa, MUS MUSCULUS
MPRGPASLLLLVLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCMQVKDRCAPVMSAFGFPWPDMLECDRFPQDNDLCIPLASSDHLLPATEEAPKVCEACKTKNEDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 2 - DNA, 885 bp, MUS MUSCULUS
atgccgcggggccctgcctcgctgctgctgctagtcctcgcctcgcactgctgcctgggctcggcgcgtgggctcttcctcttcggccagcccgacttctcctacaagcgcagcaactgcaagcccatccccgccaacctgcagctgtgccacggcatcgagtaccagaacatgcggctgcccaacctgctgggccacgagaccatgaaggaggtgctggagcaggcgggcgcctggattccgctggtcatgaagcagtgccacccggacaccaagaagttcctgtgctcgctcttcgcccctgtctgtctcgacgacctagatgagaccatccagccgtgtcactcgctctgcatgcaggtgaaggaccgctgcgccccggtcatgtccgccttcggcttcccctggccagacatgctggagtgcgaccgtttcccgcaggacaacgacctctgcatccccctcgctagtagcgaccacctcctgccggccacagaggaagctcccaaggtgtgtgaagcctgcaaaaccaagaatgaggacgacaacgacatcatggaaaccctttgtaaaaatgacttcgcactgaaaatcaaagtgaaggagataacgtacatcaacagagacaccaagatcatcctggagacaaagagcaagaccatttacaagctgaacggcgtgtccgaaagggacctgaagaaatccgtgctgtggctcaaagacagcctgcagtgcacctgtgaggagatgaacgacatcaacgctccgtatctggtcatgggacagaagcagggcggcgagctggtgatcacctccgtgaaacggtggcagaagggccagagagagttcaagcgcatctcccgcagcatccgcaagctgcaatgc

SEQ ID NO: 3 - DNA, 1859 bp, MUS MUSCULUS
  misc_feature (1059)..(1059): n is A, C, T or G
atgccgcggggccctgcctcgctgctgctgctagtcctcgcctcgcactgctgcctgggctcactagtccacgatgccgcggggccctgcctcgctgctgctgctagtcctcgcctcgcactgctgcctgggctcggcgcgtgggctcttcctcttcggccagcccgacttctcctacaagcgcagcaactgcaagcccatccccgccaacctgcagctgtgccacggcatcgagtaccagaacatgcggctgcccaacctgctgggccacgagaccatgaaggaggtgctggagcaggcgggcgcctggattccgctggtcatgaagcagtgccacccggacaccaagaagttcctgtgctcgctcttcgcccctgtctgtctcgacgacctagatgagaccatccagccgtgtcactcgctctgcatgcaggtgaaggaccgctgcgccccggtcatgtccgccttcggcttcccctggccagacatgctggagtgcgaccgtttcccgcaggacaacgacctctgcatccccctcgctagtagcgaccacctcctgccggccacagaggaagctcccaaggtgtgtgaagcctgcaaaaccaagaatgaggacgacaacgacatcatggaaaccctttgtaaaaatgacttcgcactgaaaatcaaagtgaaggagataacgtacatcaacagagacaccaagatcatcctggagacaaagagcaagaccatttacaagctgaacggcgtgtccgaaagggacctgaagaaatccgtgctgtggctcaaagacagcctgcagtgcacctgtgaggagatgaacgacatcaacgctccgtatctggtcatgggacagaagcagggcggcgagctggtgatcacctccgtgaaacggtggcagaagggccagagagagttcaagcgcatctcccgcagcatccgcaagctgcaatgctagtttcccagtggggtggcttctctccatccaggccctgagctctgtagaccacttccgctccgcgacctcatttccggtttcccaagcacagtccgggnaagctacagccccagcttggagccgcttgccctgcctcctgcatgtgtgtatccctaacatgtcctgagttataaggccctaggaggccttggaaacccatagctgttttcacggaaagcgaaaagcccatccagatcttgtacaaatattcaaactaataaaatcatgactatttttatgaagttttagaacagctcgttttaaggttagttttgaatagctgtagtactttgacccgaggggcattttctctctttggtcagtctgttggcttataccgtgcacttaggttgccatgtcaggcgaattgtttctttttttttttttttttccctctgtggtctaagcttgtgggtcccagacttagttgagataaagctggctgttatctcaaagtcttcctcagttccagcctgagaatcggcatctaagtcttcaaacatttcgttgctcgttttatgccctcatgagctctgaccattgcatgcgttcccatcccagctacagaacttcagtttataagcacacagtaaccattcctcattgcatgatgccctcaaataaaaagtgaatacagtctataaattgacgagtattttaagctttgtttaaaacatcttttaattcaattttttaatcattttttttgcaaactaaatcattgtagcttacctgtaatatacgtagtagttgacctggaaaagttgtaaaaatattgctttaaccgacactgtaaatatttcagataaacattatattctttgtatataaactttaaaaaaaaaaaaaaaaaaaaaaa

SEQ ID NO: 4 - DNA, 1799 bp, MUS MUSCULUS
  CDS (14)..(898)
tcactagtccacgatgccgcggggccctgcctcgctgctgctgctagtcctcgcctcgcactgctgcctgggctcggcgcgtgggctcttcctcttcggccagcccgacttctcctacaagcgcagcaactgcaagcccatccccgccaacctgcagctgtgccacggcatcgagtaccagaacatgcggctgcccaacctgctgggccacgagaccatgaaggaggtgctggagcaggcgggcgcctggattccgctggtcatgaagcagtgccacccggacaccaagaagttcctgtgctcgctcttcgcccctgtctgtctcgacgacctagatgagaccatccagccgtgtcactcgctctgcatgcaggtgaaggaccgctgcgccccggtcatgtccgccttcggcttcccctggccagacatgctggagtgcgaccgtttcccgcaggacaacgacctctgcatccccctcgctagtagcgaccacctcctgccggccacagaggaagctcccaaggtgtgtgaagcctgcaaaaccaagaatgaggacgacaacgacatcatggaaaccctttgtaaaaatgacttcgcactgaaaatcaaagtgaaggagataacgtacatcaacagagacaccaagatcatcctggagacaaagagcaagaccatttacaagctgaacggcgtgtccgaaagggacctgaagaaatccgtgctgtggctcaaagacagcctgcagtgcacctgtgaggagatgaacgacatcaacgctccgtatctggtcatgggacagaagcagggcggcgagctggtgatcacctccgtgaaacggtggcagaagggccagagagagttcaagcgcatctcccgcagcatccgcaagctgcaatgctagtttcccagtggggtggcttctctccatccaggccctgagctctgtagaccacttccgctccgcgacctcatttccggtttcccaagcacagtccgggnaagctacagccccagcttggagccgcttgccctgcctcctgcatgtgtgtatccctaacatgtcctgagttataaggccctaggaggccttggaaacccatagctgttttcacggaaagcgaaaagcccatccagatcttgtacaaatattcaaactaataaaatcatgactatttttatgaagttttagaacagctcgttttaaggttagttttgaatagctgtagtactttgacccgaggggcattttctctctttggtcagtctgttggcttataccgtgcacttaggttgccatgtcaggcgaattgtttctttttttttttttttttccctctgtggtctaagcttgtgggtcccagacttagttgagataaagctggctgttatctcaaagtcttcctcagttccagcctgagaatcggcatctaagtcttcaaacatttcgttgctcgttttatgccctcatgagctctgaccattgcatgcgttcccatcccagctacagaacttcagtttataagcacacagtaaccattcctcattgcatgatgccctcaaataaaaagtgaatacagtctataaattgacgagtattttaagctttgtttaaaacatcttttaattcaattttttaatcattttttttgcaaactaaatcattgtagcttacctgtaatatacgtagtagttgacctggaaaagttgtaaaaatattgctttaaccgacactgtaaatatttcagataaacattatattctttgtatataaactttaaaaaaaaaaaaaaaaaaaaaaa

SEQ ID NO: 5 - protein, 295 aa, MUS MUSCULUS; identical to SEQ ID NO: 1
  misc_feature (999)..(999): n is A, T, C or G

SEQ ID NO: 6 - protein, 127 aa, DROSPHILA
DGLPHHNRCEPITISICKNIPYNMTIMPNLIGHTKQEEAGLEVHQFAPLVKIGCSDDLQLFLCSLYVPVCTILERPIPPCRSLCESARVCEKIMKTYNFNWPENLECSKFPVHGGEDLCVAENTTSS

SEQ ID NO: 7 - DNA, 105 bp, Artificial Sequence
  multi-cloning sites of the pcD-SRalpha296 vector
ctgcagtaatacgactcactataggggagagctcgtcgacagatctgaattccatatgcccggggcggccgcactagtctatagtgtcacctaaatcgtggtacc

SEQ ID NO: 8 - DNA, 40 bp, Artificial Sequence
  a single stranded oligo DNA (17mer dC) for synthesis of a double-stranded cDNA described in Example 3
gatgcggccgcgtcgacgaattcccccccccccccccccc

SEQ ID NO: 9 - DNA, 29 bp, Artificial Sequence
  single stranded oligo DNA (lone linker) linked with double-stranded cDNA described in Example 3
gaggtacaagcttgatatcgagctcgcgg

SEQ ID NO: 10 - DNA, 26 bp, Artificial Sequence
  complemental oligo DNA to single stranded oligo DNA (lone linker) shown in Seq. No. 9
ccgcgagctcgatatcaagcttgtac

SEQ ID NO: 11 - DNA, 23 bp, Artificial Sequence
  synthetic primer (NLC) for PCR reactions using synthesized double-stranded cDNA as a template
gatgcggccgcgtcgacgaattc

SEQ ID NO: 12 - DNA, 29 bp, Artificial Sequence
  primer (LLHES) for PCR reactions using synthesized double-stranded cDNA as a template
gaggtacaagcttgatatcgagctcgcgg

SEQ ID NO: 13 - DNA, 25 bp, Artificial Sequence
  DNA sequencing primer specific for pUSCST alphaML2-hTac vector
gcctgtacggaagtgttacttctgc

SEQ ID NO: 14 - DNA, 20 bp, Artificial Sequence
  DNA sequencing primer specific for pUSCST alphaML2-hTac vector
ccatggctttgaatgtggcg

(19) United States
(12) Patent Application Publication
(10) Pub. No.: US 2002/0165350 A1
(43) Pub. Date: Nov. 7, 2002

(54) Novel polypeptide, a method of producing it, and utility of the polypeptide

(76) Inventors: Tasuku Honjo, Kyoto (JP); Michio Shirozu, Kyoto (JP); Hideaki Tada, Mishima-gun (JP)

Correspondence Address: STEVENS, DAVIS, MILLER & MOSHER, L.L.P., Suite 850, 1615 L Street, N.W., Washington, DC, 20036, US

(21) Appl. No.: 09/934,483

(22) Filed: Aug. 23, 2001

Related U.S. Application Data
(63) Continuation of application No. 09/308,038, filed on Jul. 16, 1999, now abandoned.
Application No. 09/308,038 is a 371 of international application No. PCT/JP97/03953, filed on Oct. 30, 1997.

(30) Foreign Application Priority Data
Nov. 13, 1996 (JP) 8-301666

(51) Int. Cl. A61K 38/17; C07K 14/435; C12P 21/02; C12N 5/06; C07K 16/40

(52) U.S. Cl. 530/350; 530/388.1; 435/69.1; 435/325; 435/320.1; 514/12

(57) ABSTRACT

A polypeptide comprising 295 amino acids which is produced by mouse bone marrow stromal cells, a method of producing the polypeptide, a DNA encoding the polypeptide, a fragment which selectively hybridized the DNA sequence, a replication or expression vector comprising the DNA, a host cell transfected with the vector, an antibody of the peptide, a pharmaceutical composition containing the polypeptide or the antibody.

### FIELD OF THE INVENTION

The present-invention provides a novel polypeptide, a method for preparation of it, a DNAs encoding it, a vector carrying the DNA, a host cell transformed with the vector, an antibody against the polypeptide, a pharmaceutical composition containing the polypeptide or the antibody.

More particularly, the present invention provides a novel polypeptide which is produced by a certain mouse stromal cell line, preparation of the polypeptide, DNA encoding the polypeptide, a-vector carrying the DNA, a host cell transformed by the vector, an antibody of the polypeptide, a pharmaceutical composition containing the polypeptide or the antibody.

### BACKGROUND OF THE INVENTION

It is known that bone marrow stromal cells form bone marrow micro environment of immunologic, hematopoietic system etc, and they produce and secret essential factors to induce of proliferation and differentiation of stem cells, e.g. IL-7, SCF, IL-11, M-CSF, G-CSF, GM-CSF, IL-6, TGF-β, LIF etc. It is also made clear that a certain bone marrow stromal cells are related to bone metabolism (Kenneth Dorshkind Annu. Rev. Immunol. 8, 111-137. 1990). However, roles of stromal cell are not reconstituted completely from only isolated factors yet. It may suggest that existence of any factors which are not isolated yet.

### DISCLOSURE OF THE INVENTION

The present inventors have directed their attention to this point and energetic research has been carried out in order to find novel factors (polypeptides) especially secretory and membrane protein which are generated by a certain stromal cells.

Until now, when a man skilled in the art intends to obtain a particular polypeptide or a DNA encoding it, he generally utilizes methods by confirming an intended biological activity in a tissue or in a cell medium, isolating and purifying the polypeptide and then cloning a gene or methods by “expression-cloning” with the guidance of the biological activity.

However, physiologically active polypeptides in living body have often many kinds of activities. Therefore, it is increasing that after a gene is cloned, the gene is found to be identical to that encoding a polypeptide already known. Generally glia generates only a very slight amount of a factor and it makes difficult to isolate and to purify the factor and to confirm its biological activity.

Recent rapid developments in techniques for constructing cDNAs and sequencing techniques have made it possible to quickly sequence a large amount of cDNAs. By utilizing these techniques, a process, which comprises constructing cDNAs at random, identifying the nucleotide sequences thereof, expressing novel polypeptides encoded by them, is now in progress. Although this process is advantageous in that a gene can be cloned and information regarding its nucleotide sequence can be obtained without any biochemical or genetic analysis, the target gene can be discovered thereby only accidentally in many cases.

The present inventors have studied cloning method of genes coding proliferation and/or differentiation factors functioning in hematopoietic systems and immune systems. Focusing their attention on the fact that most of the secretory proteins such as proliferation and/or differentiation factors (for example various cytokines) and membrane proteins such as receptors thereof (hereafter these proteins will be referred to generally as secretory proteins and the like) have sequences called signal peptides in the N-termini, the inventors conducted extensive studies on a process for efficiently and selectively cloning a gene coding for a signal peptide. Thus, we have found a screening method for the existence or absence of signal peptide easily by effective amplification of N-termini fragment (see Japanese Patent Kokai No. 6-315380). We have succeeded to find out a novel factor (peptide) which is produced by bone marrow stromal cells and a DNA encoding the said peptide by the method and then achieved the present invention.

There was no polypeptide having amino acid sequence which is identical to the polypeptide of the present invention, when amino acid sequences of the polypeptide was compared by a computer to all known sequences in data base of Swiss Prot Release 33. The polypeptide of the present invention was found to show significant homology to each extracellular Cys-rich region of Drosophila Frizzled protein and mammalian counter parts. So it was confirmed that the polypeptide of the present invention is a novel secretory protein containing Frizzled Cys-rich motif.

The present invention provides:

1) Substantially purified form of the polypeptide comprising the amino-acid sequence shown in SEQ ID NO. 1, homologue thereof, fragment thereof or homologue of the fragment.

2) A polypeptide, according to item 1, comprising the amino-acid sequence shown in SEQ ID NO. 1.

3) A DNA encoding the polypeptide according to item 2.

4) A DNA, according to item 3, comprising the nucleotide sequence shown in SEQ ID NO. 2 or a fragment selectively hybridized to the DNA.

5) A DNA, according to item 3, comprising the nucleotide sequence shown in SEQ ID NO. 3 or a fragment selectively hybridized to the DNA.

6) A replication or expression vector carrying the DNA according to items 3 to 5.

7) A host cell transformed with the replication or expression vector according to item 6.

8) A method for producing the polypeptide according to claim 1 or 2 which comprises culturing a host cell according to claim 7 under a condition effective to express the polypeptide according to item 1 or 2.

9) A monoclonal or polyclonal antibody against the polypeptide according to item 1 or 2.

10) A pharmaceutical composition containing the polypeptide according to item 1 or 2 or the antibody according to claim 9, in association with pharmaceutically acceptable diluent and/or carrier.

### BRIEF DESCRIPTION OF THE DRAWING

FIG. 1 shows the conception of the preparation of cDNA library.

FIG. 2 shows the homology of amino acid sequences between mouse SDF5 and Cys-rich domain of Drosophila Frizzled protein (d-Fz).

FIG. 3 shows the construction of plasmid vector of pUCSR α ML2.

FIG. 4 shows the construction of pUCSR α ML2-hTac.

### DETAILED DESCRIPTION OF THE INVENTION

The present invention provides:

1) a polypeptide comprising an amino acid sequence shown in SEQ ID NO. 1,

2) a DNA encoding the polypeptide described above 1),

3) a DNA comprising an nucleotide sequence shown in SEQ ID NO. 2,

4) a DNA comprising an nucleotide sequence shown in SEQ ID NO. 3.

The present invention provides a polypeptide comprising the amino acid sequence shown in SEQ ID NO. 1 in substantially purified form, a homologue thereof, a fragment of the sequence and a homologue of the fragment.

Further, the present invention is concerned with a DNA encoding the above peptide. More particularly the present invention is provided DNA comprising the nucleotide sequence shown in SEQ ID NO. 2 or 3, and DNA containing a fragment which is selectively hybridizing to the DNA comprising nucleotide sequence shown in SEQ ID NO. 2 or 3.

A polypeptide of SEQ ID NO. 1 in substantially purified form will generally comprise the polypeptide in a preparation in which more than 90%, e.g. 95%, 98% or 99% of the polypeptide in the preparation is that of the SEQ ID NO. 1.

A polypeptide homologue of the SEQ ID NO. 1 will be generally at least 70%, preferably at least 80 or 90% and more preferably at least 95% homologous to the polypeptide of SEQ ID NO. 1 over a region of at least 20, preferably at least 30, for instance 40, 60 or 100 more contiguous amino acids. Such a polypeptide homologue will be referred to a polypeptide of the present invention.

Generally, a fragment of SEQ ID NO. 1 or its homologues will be at least 10, preferably at least 15, for example 20, 25, 30, 40, 50 or 60 amino acids in length, and are also referred to by the term “a polypeptide of the present invention”.

A cDNA capable of selectively hybridizing to the DNA of SEQ ID NO. 2 or 3 will be generally at least 70%, preferably at least 80 or 90% and more preferably at least 95% homologous to the DNA of SEQ ID NO. 2 or 3 over a region of at least 20, preferably at least 30, for instance 40, 60 or 100 or more contiguous nucleotides. Such DNA will be referred to “a cDNA of the present invention”.

Fragments of the DNA of SEQ ID NO. 2 or 3 will be at least 15, preferably at least 20, for example 25, 30 or 40 nucleotides in length, and will be also referred to “a DNA of the present invention” as used herein.

A further embodiment of the present invention provides replication and expression vectors carrying DNA of the invention. The vectors may be, for example, plasmid, virus or phage vectors provided with an origin of replication, optionally a promoter for the expression of the said DNA and optionally a regulator of the promoter. The vector may contain one or more selectable marker genes, for example a ampicillin resistance gene. The vector may be used in vitro, for example of the production of RNA corresponding to the DNA, or used to transfect or transfect a host cell.

A further embodiment of the present invention provides host cells transformed with the vectors for the replication and expression of the cDNA of the invention, including the DNA SEQ ID NO. 2 or 3 or the open reading frame thereof. The cells will be chosen to be compatible with the vector and may for example be bacterial, yeast, insect or mammalian.

A further embodiment of the present invention provides a method of producing a polypeptide which comprises culturing host cells of the present invention under conditions effective to express a polypeptide of the invention. Preferably, in addition, such a method is carried out under conditions in which the polypeptide of the invention is expressed and then produced from the host cells.

cDNA of the present invention may also be inserted into the vectors described above in an antisense orientation in order to proved for the production of antisense RNA. Antisense RNA may also be produced by synthetic means. Such antisense RNA may be used in a method of controlling the levels of a polypeptide of the invention in a cell.

The invention also provides monoclonal or polyclonal antibodies against a polypeptide of the invention. The invention further provides a process for the production of monoclonal or polyclonal antibodies to the polypeptides of the invention. Monoclonal antibodies may be prepared by common hybridoma technology using polypeptides of the invention or fragments thereof, as an immunogen. Polyclonal antibodies may also be prepared by common means which comprise inoculating host animals, for example a rat or a rabbit, with polypeptides of the invention and recovering immune serum. The present invention also provides pharmaceutical compositions containing a polypeptide of the invention, or an antibody thereof, in association with a pharmaceutically acceptable diluent and/or carrier.

The polypeptide (1) of the present invention includes that which a part of their amino acid sequence is lacking (e.g., a polypeptide comprised of the only essential sequence for revealing a biological activity in an amino acid sequence shown in SEQ ID NO. 1), that which a part of their amino acid sequence is replaced by other amino acids (e.g., those replaced by an amino acid comprising a similar property) and that which other amino acids are added or inserted into a part of their amino acid sequence, as well as those comprising the amino acid sequence shown in SEQ ID NO. 1.

As known well, there are one to six kinds of codon as that encoding one amino acid (for example, one kind of codon for Methioine (Met), and six kinds of codon for leucine (Leu) are known). Accordingly, the nucleotide sequence of DNA can be changed in order to encode the polypeptide comprising the same amino acid sequence.

The DNA of the present invention, specified in (2) includes a group of every nucleotide sequences encoding polypeptides (1). shown in SEQ ID NO. 1. There is a probability that yield of a polypeptide is improved by changing a nucleotide sequence.

The cDNA specified in (3) is the embodiment of the cDNA shown in (2), and indicate the sequence of natural form.

The cDNA shown in (4) indicates the sequence of the cDNA specified in (3) with natural non-translational region.

DNA carrying nucleotide sequence shown in SEQ ID NO. 3 is prepared by the following method:

Preparation of cDNA library of signal peptide

(1) mRNAs were isolated from target cells, a first strand DNA corresponded to the mRNA obtained was synthesized with random primer, oligo dG was added to 3′-end of the first strand,

(2) A second strand DNA for the first strand was synthesized by using poly oligomer which is linked a specific restriction enzyme site (enzyme I) as primer, to obtain double strand,

(3) Double stranded DNA obtain in (2) was digested, fractionated by fragment size, fragments are linked a linker containing a specific restriction enzyme site (enzyme II) which is not same enzyme site I, and fractionated,

(4) PCR is performed with a primer having enzyme site I and a primer having enzyme site II, amplified cDNAs are digested at enzyme I-enzyme II, and fractionated,

(5) cDNA fragments are ligated upstream of the gene of known secretory protein which is deleted signal peptide, the fragments are inserted into plasmid vector for expression in eukaryotic cell, eukaryotic cell are transformed.

Explained in detail, step (1) isolation of mRNA may be carried out in accordance with the method of Okayama, H et al (described in Method in Enzymology, vol. 154, 3 (1987)) after target cell line is stimulated by a proper stimulant. Any cells may be used having possibility to produce secretory protein etc. Example of such cells are, for example, neuronal cells, hematopoietic cells. Synthesis of first single strand cDNA may be carried out by known methods. Random primer in the market may be used. Oligo dC is added to 3′-end of the first single cDNA strand with terminal deoxytransferase.

In step (2) synthesis of double strand DNA may be carried out by known methods. Restriction enzyme (enzyme I) site which is linked to poly oligomer (primer) and restriction enzyme (enzyme II) site may be used any enzymes, except for the enzymes used are same one. Preferably, SalI is used as enzyme I, SacI is used as enzyme II.

In step (3), cDNAs are fragmented to 500 bp at average by ultrasonication, fragments are fractionated to 400-800 bp with agarose gel electrophoresis (AGE), blunted the ends with T4 DNA polymerase, linked enzyme II adapter and fractionated to 400-800 bp DNA with agarose gel electrophoresis. Enzyme II is any one may be used as described before. In this step, possibility of existence of cDNA containing signal peptide between enzyme I and enzyme II will be up.

In step (4), PCR is carried out in order to further amplify a fragment located between the enzymes I and II, in which a signal peptide may be encoded. PCR is a well-known technique and some automated devices for the aim are commercially available. It is sufficient to amplify 25-30 times. The cDNA thus amplified is digested with enzyme I and II and is subjected to electrophoresis on an agarose gel to fractionated into fragmented cDNAs of 400 to 800 bp.

In step (5), transformation is performed. Gene of known secretory protein (it is called reporter gene) which is deleted the signal peptide is inserted to a plasmid vector for expression in eukaryotic cell. Many kinds of plasmid vectors for eukaryotic cell are known. For example, pcDL-SR α and pcEV-4 which are work in Escherichia coli are used.

For reporter gene, many kinds of gene of mature protein part of soluble secretory protein and membrane protein are used. Reporter gene used must selected which may be confirmed the expression with some method, for example, antibody method.

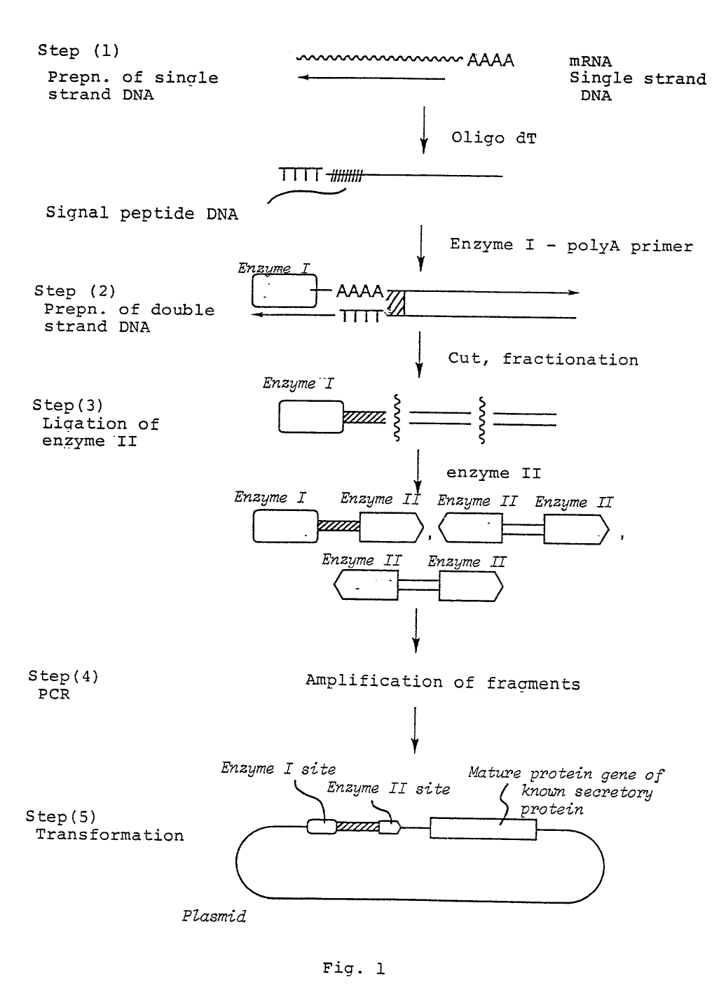

Preferably, gene of human IL-2 receptor α. Many host Escherichia coli strains for transformation are known, any strain may be used, DH5 competent cell is preferable_isee Gene, 96, 23 (1990)). Transformant is cultured by known methods to obtain cDNA library of the present invention (the conception shows in FIG. 1.).

In the process for constructing a cDNA library according to the present invention, there is a high possibility that gene fragments coding for signal peptides are contained in the library. However, not every clone contains said fragment. Further, not all of the gene fragments code for unknown (novel) signal peptides. It is therefore necessary to screen a gene fragment coding for an unknown signal peptide from said library.

Namely, the cDNA library is divided into small pools of an appropriate size and integrated into an expression system. Examples of the expression system for producing a polypeptide include mammalian cells (for example, monkey COS-7 cells, Chinese hamster CHO cells, mouse L cells etc.). Transfection may be performed in accordance with well known methods such as the DEAE-dextran method. After the completion of the incubation, the expression of the reporter gene is examined.

It is known that a reporter gene would be expressed even though the signal peptide is the one characteristic to another secretory protein. That is to say, the fact that the reporter gene has been expressed indicates that a signal peptide of same secretory protein has been integrated into the library. Positive pools are further divided into smaller ones and the expression and the judgement are repeated until a single clone is obtained. The expression of the reporter gene can be judged by, for example, fluorescence-labeled antibody assay, enzyme-linked immunosorbent assay (ELISA) or radio-immuno assay (RIA), depending on kinds of the employed reporter gene.

Next, the nucleotide sequence of the isolated positive clone is determined. In the case of a cDNA which is proved to code for an unknown protein, the clone of the full length is isolated with the use of the cDNA as a probe and the full nucleotide sequence can be thus identified. All of these operations are carried out by methods which are well known by those skilled in the art. For example, the nucleotide sequence may be identified by Maxam-Gilbert method or the dideoxy terminator method. On the other hand, the full length may be sequenced in accordance with a method described in Molecular Cloning (Sambrook, J., Fritsch, E. F. and Maniatis, T., Cold Spring Harbor Laboratory Press (1989)).

Once the nucleotide sequences shown in SEQ ID NO. 2 or 3 are determined partially or preferably fully, it is possible to obtain DNA encodes mammalian protein of the present invention itself, homologue or subset. cDNA library or mRNA derived from mammals was screened by PCR with any synthesized oligonucleotide primers or by hybridization with an appropriate mouse nucleotide fragment as a probe. It is possible to obtain cDNA encodes other mammalian homologue protein from other mammalian cDNA or genome library.

It is necessary to confirm that cDNA obtained covers full or almost full length of intact mRNA. These confirmation may be carried out by Northern analysis using the cDNA as a probe (see Molecular Cloning). If the size of mRNA obtained from the hybridized band and size of the cDNA are almost same, it will be thought that the cDNA is almost full length.

Once the nucleotide sequence shown in SEQ ID NO. 2 or 3 are determined, DNA of the present invention may be obtained by chemical synthesis, by hybridization making use of a fragment of DNA of the present invention as a probe. Furthermore, DNA of the present invention may be obtained in a desired amount by transforming with a vector DNA inserted a DNA of the present invention into a proper host, followed by culturing the transformant.

The polypeptides of the present invention may be prepared by:

(1) isolating and purifying from an organism or a cultured cell,

(2) chemically synthesizing, or

(3) using recombinant DNA technology, preferably, by the method described in (3).

Examples of expression system for producing a polypeptide by using recombinant DNA technology are the expression systems of bacteria, yeast, insect cells and mammalian cells.

In the expression of the polypeptide, for example, in Escherichia coli, the expression vector is prepared by adding the initiation codon (ATG) to 5′ end of a DNA encoding mature peptide, connecting the DNA thus obtained to the downstream of a proper promoter (e.g., trp promoter, lac promoter, λPL promoter, T7 promoter etc.), and then inserting it into a vector (e.g., pBR322, pUC18, pUC19 etc.) which functions in an Escherichia Coli strain.

Then, an Escherichia coli strain (e.g., Escherichia coli DH1 strain, Escherichia coli JM109 strain, Escherichia coli HB101 strain, etc.) which is transformed with the expression vector described above may be cultured in a appropriate medium to obtain the desired polypeptide. When a signal peptide of bacteria (e.g., signal peptide of pel B) is utilized, the desired polypeptide may be also released in periplasm. Furthermore, a fusion protein with other polypeptide may be also produced easily.

Furthermore, the expression in a mammalian cell may be carried out, for example, by inserting the DNA shown in SEQ ID NO. 3 into the downstream of a proper promoter (e.g., SV40 promoter, LTR promoter, metallothionein promoter etc.) in a proper vector (e.g., retrovirus vector, papilloma virus vector, vaccinia virus vector, SV40 vector, etc.) to obtain an expression vector, and transforming a proper mammalian cell (e.g., monkey COS-7 cell, Chinese hamster CHO cell, mouse L cell etc.) with the expression vector thus obtained, and then culturing the transformant in a proper medium to get a desired polypeptide in the culture medium. The polypeptide thus obtained may be isolated and purified by conventional biochemical methods.

### Possibility for the Industrial Usage

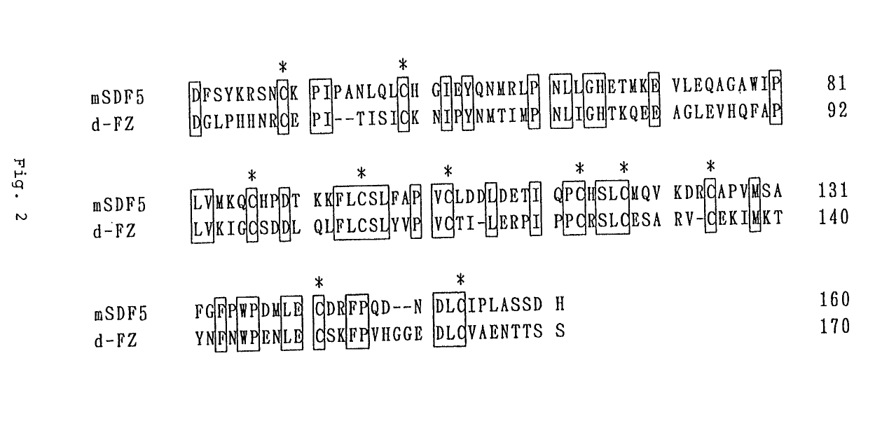

The polypeptide of the present invention has significantly homologous to Frizzled protein (Charles R. Vinson et al., Nature, 338, 263-264 (1989), Yanshu Wang et al, J. Biol. Chem., 271, 4468-4476 (1996). Ten cystein residues (Cys-rich motif) which exists in extracellular domain of Frizzled protein are conserved in the polypeptide of the present invention (see FIG. 2).

It is known that Frizzled protein is a receptor protein having seven transmembrane regions and it plays an important role in morphology (especially decision of the polarity of the tissue) and Cys-rich motif in N-termini is a binding region to the ligand (Purnima et al, Nature, 382, 225-230 (1996)). That is, mouse SDF-5 protein of the present invention is expected to bind to proteins such as Wnts and proteins which is thought to bind the extracellular domain of Frizzled protein family. It can be considered that the polypeptide of the present invention may show function by regulating the binding of Wnt protein to a receptor such as a member of Frizzled protein family, as an embodiment of the present invention.

Therefore, it is expected that the polypeptide of the present invention may be involved in the formation of shapes via Cys-rich motif, and it may be predicted to possess an activity related to the formation and repair and so on of each organism and tissue.

Further, it is thought that the polypeptide of the present invention relates the differentiation, proliferation, growth and bio-activity which relates to the survival of hematopoietic cells, bio-activity which relates to immune system, differentiation and proliferation of tumors and bio-activity which relates to inflammatory or bio-activity of bone metabolism, as the polypeptide is produced and secreted by a stromal cell line.

The polypeptide of the present invention has a possibility to have following functions by itself or interaction with other cytokines. For example, promotion of differentiation from mesenchymal stem cells to osteoblasts or chondrocytes and promotion of bone absorption by activation or chonrdocytes and promotion of bone absorption by activation of osteoclasts and promotion of differentiation from monocytes to osteoclasts.

Further, proliferation of B-cells, T-cells and mast cells or class specific induction by promotion of class switch of immunoglobulin, differentiation of B-cells to antibody-forming cells, proliferation and differentiation of precursors of granulocytes, proliferation and differentiation of precursors of monocytes-macrophages, proliferation of precursors of megakaryocytes, proliferation and differentiation of precursors of neutrophils, proliferation and differentiation of precursors of B- or T-cells, promotion of production of erythrocytes, sustainment of proliferation of erythrocytes, neutrophils, eosinophils, basophils, monocytes-macrophages, mast cells, precursors of megakaryocytes, promotion of migration of neutrophils, monocytes-macrophages, B- or T-cells, proliferation of thymocytes, suppression of differentiation of adipocytes, proliferation of natural killer cells, proliferation of hematopoietic stem cells, suppression of proliferation of stem cells and each precursors of hematopoietic cells.

As a certain Frizzled protein relates to the generation of nerve system cells, it is expected that the polypeptide may act to nerve system. So the polypeptide may possess an activity on differentiation and survival of each nerve cells which act by neurotransmitter, accelerating activity of proliferation of glial cells, extension activity of nerurites, survival of neuroganglion cells, proliferation and differentiation activity of astrocytes, proliferation activity of peripheral nerve cells, proliferation activity of Schwann cells, proliferation and survival activity of motorial cells.

Furthermore, in the process of development of early embryonic, this polypeptide is expected to promote or inhibit the organogenesis of epidermis, brain, backbone, and nervous system by induction of ectoderm, that of notochord connective tissues(bone, muscle, tendon), hemocytes, heart, kidney, and genital organs by induction of mesoderm, and that of digestive apparatus (stomach, intestine, liver, pancreas), respiratory apparatus (lung, trachea) by induction of endoderm. In a body, also, this polypeptide is thought to proliferate or inhibit the above organs.

Therefore, this polypeptide itself is expected to be used as an agent for the prevention or treatment of disease of progression or suppression of immune, nervous, or bone metabolic function, hypoplasia or overgrowth of hematopoietic cells: inflammatory disease (rheumatism, ulcerative colitis, etc.), decrease of hematopoietic stem cells after bone marrow transplantation, decrease of leukocytes, platelets, B-cells, or T-cells after radiation exposure or chemotherapeutic dosage against cancer or leukemia, anemia, infectious disease, cancer, leukemia, AIDS, various degenerative disease (Alzheimer's disease, multiple sclerosis, etc.), or nervous lesion.

In addition, since this polypeptide is thought to induce the differentiation or growth of organs derived from ectoderm, mesoderm, and endoderm, this polypeptide is expected to be an agent for tissue repair (epidermis, bone, muscle, tendon, heart, kidney, stomach, intestine, liver, pancreas, lung, and trachea, etc.).

Further, polyclonal or monoclonal antibody against the polypeptide of the present invention can be used in the determination of the amount of the said polypeptide in organism, and thereby, may be utilized for the purpose of investigating the relationship between the said polypeptide and diseases, or for the purpose of diagnosing diseases, and the like. Polyclonal and monoclonal antibody thereof may be prepared by conventional methods by using the said polypeptide or the fragment thereof as an antigen.

Further, purification of the protein (receptor) which binds to the said peptide and molecular cloning of the gene may be performed by using the said protein. It also may be used the detection of agonist or antagonist of the said polypeptide.

The DNA of the present invention may be utilized as an important and essential template in preparing the polypeptide of the present invention which is expected to possess various use or for diagnosis of and in the treatment of gene diseases (the treatment of gene defect disease and the treatment by inhibiting expression of the polypeptide by antisense DNA (RNA), and the like). Further, genomic DNA may be isolated by using the DNA of the present invention as a probe. Similarly, it is possible to isolate genes having high homology to the DNA of the present invention in human or those of other species.

For the prevention and treatment of hypoplasia or overgrowth of hematopoietic cells, diseases of progression or suppression of nervous, diseases of progression or suppression of immune, for example, inflammatory disease (rheumatism, ulcerative colitis, etc.), decrease of hematopoietic stem cells after bone marrow transplantation, decrease of leukocytes, platelets, B-cells, or T-cells after radiation exposure or chemotherapeutic dosage against cancer or leukemia, anemia, infectious disease, cancer, leukemia, AIDS, various degenerative disease (Alzheimer's disease, multiple sclerosis etc.) or nerve lesion, for the prevention and treatment of abnormal bone metabolism (osteoporosis etc.) or repair of tissue, the polypeptide of the invention may be administered systemically or partially in most cases, usually by oral or parenteral administration, preferably orally, intravenously or intraventricularly.

The doses to be administered are determined depending upon age, body weight, symptom, the desired therapeutic effect, the route of administration, and the duration of the treatment etc. In the human adult, the doses per person per dose are generally between 100 μg and 100 mg, by oral administration, up to several times per day, and between 10 μg and 100 mg, by parenteral administration up to several times per day.

As mentioned above, the doses to be used depend upon various conditions. Therefore, there are cases in which doses lower than or greater than the ranges specified above may be used.

The compounds of the present invention, may be administered as solid compositions, liquid compositions or other compositions for oral administration, as injections, liniments or suppositories etc. for parenteral administration.

Solid compositions for oral administration include compressed tablets, pills, capsules, dispersible powders, granules. Capsules include soft capsules and hard capsules.

In such compositions, one or more of the active compound(s) is or are admixed with at least one inert diluent (such as lactose, mannitol, glucose, hydroxypropyl cellulose, microcrystalline cellulose, starch, polyvinylpyrrolidone, magnesium metasilicate aluminate, etc.). The compositions may also comprise, as is normal practice, additional substances other than inert diluents: e.g. lubricating agents (such as magnesium stearate etc.), disintegrating agents (such as cellulose calcium glycolate, etc.), stabilizing agents (such as human serum albumin, lactose etc.), and assisting agents for dissolving (such as arginine, asparaginic acid etc.).

The tablets or pills may, if desired, be coated with a film of gastric or enteric material (such as sugar, gelatin, hydroxypropyl cellulose or hydroxypropylmethyl cellulose phthalate, etc.), or be coated with more than two films. And further, coating may include containment within capsules of absorbable materials such as gelatin.

Liquid compositions for oral administration include pharmaceutically-acceptable emulsions, solutions, syrups and elixirs. In such compositions, one or more of the active compound(s) is or are contained in inert diluent(s) commonly used in the art (purified water, ethanol etc.). Besides inert diluents, such compositions may also comprise adjuvants (such as wetting agents, suspending agents, etc.), sweetening agents, flavoring agents, perfuming agents, and preserving agents.

Other compositions for oral administration included spray compositions which may be prepared by known methods and which comprise one or more of the active compound(s). Spray compositions may comprise additional substances other than inert diluents: e.g. stabilizing agents (sodium sulfite etc.), isotonic buffer (sodium chloride, sodium citrate, citric acid, etc.). For preparation of such spray compositions, for example, the method described in the U.S. Pat. No. 2,868,691 or 3,095,355 (herein incorporated in their entireties by reference) may be used.

Injections for parenteral administration include sterile aqueous or non-aqueous solutions, suspensions and emulsions. In such compositions, one or more active compound(s) is or are admixed with at least one inert aqueous diluent(s) (distilled water for injection, physiological salt solution, etc.) or inert non-aqueous diluents(s) (propylene glycol, polyethlene glycol, olive oil, ethanol, POLYSOLBATE 80 TM, etc.).

Injections may comprise additional other than inert diluents: e.g. preserving agents, wetting agents, emulsifying agents, dispersing agents, stabilizing agent (such as human serum albumin, lactose, etc.), and assisting agents such as assisting agents for dissolving (arginine, asparaginic acid, etc.).

They may be sterilized for example, by filtration through a bacteria-retaining filter, by incorporation of sterilizing agents in the compositions or by irradiation. They may also be manufactured in the form of sterile solid compositions, for example, by freeze-drying, and which can be dissolved in sterile water or some other sterile diluents for injection immediately before used.

Other compositions for parenteral administration include liquids for external use, and endermic liniments (ointment, etc.), suppositories for rectal administration and pessaries which comprise one or more of the active compound(s) and may be prepared by known methods.

### The Best Mode for the Enforcement

The following examples are illustrated, but not limit, the present invention.

### EXAMPLE 1

### Construction of Plasmid for Expression Vector

pc D-SR α 296 vector_iMol. Cell. Biol., 8, 966 (1988)) which are prepared by Takebe et al is a superior vector having promoter system (SR α) which is constructing SV40 initiation promoter, R region of LTR of HTLV-I and a part of U5 sequence. But, it have following demerits: (1) Its cloning site for insertion is only one EcoRI, (2) Yield of the vector recovery is low as pBR322 vector is used as vector region. So, we prepared modified pcD-SR α 296 vector using pUC19 vector as a template which has multi cloning sites for insertion as follows:

pcD-SR α 296 vector (presented by Dr. Takebe of National Institute of Health) was digested at SalI, 1.7 kb fragments including SR a promoter were isolated and recovered with agarose electrophoresis. The fragments were created blunt-ends by Klenow treatment.

After pUC19 vector was digested at NdeI and Hind III, the digested 2.4 kbp fragment including AmpR and pUCori regions was purified with agarose electrophoresis, and created blunt-ends by Klenow treatment and followed by dephospholilation on 5′ termini by BAP (bacterial alkali phosphatase).

1.7 kbp fragments including SR a promoter and 2.4 kbp fragments including pUCori were made circle by ligation to obtain new vector. The vector obtained was removed PsdI-KpnI fragment and replaced synthesized the following polylinker including T7 and SP6 promoter.

```
CTGCAGTAATACGACTCACTATAGGGGAGAGCTCGTCGACAGATCTGAATTCCATATGCCCGGGGCGGCCGC
  PST T7 promoter Sac I Not I
                                                                    \overline{ Sal I} \overline{ Sma I}
                                                                                \overline{Bgl II} \overline{ Nde I}
                                                                                            \overline{ EcoRI}
ACTAGTCTATAGTGTCACCTAAATCGTGGTACC
  Spe I SP 6 promoter Kpn I
```

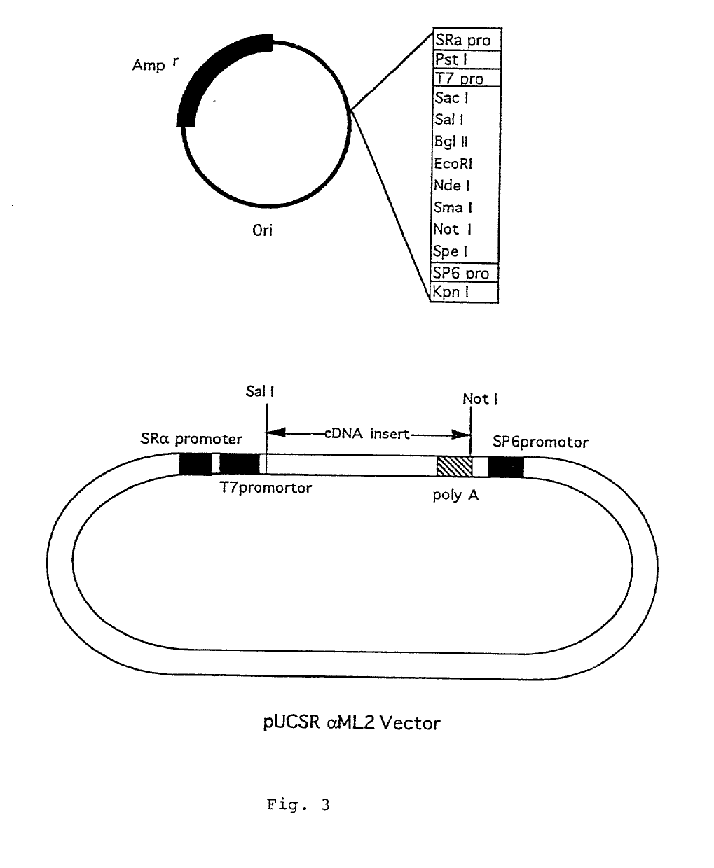

The plasmid vector (ca 3.9 kbp, shown in FIG. 3.) was named pUCSR α ML2.

The pUCSR α ML2 has the following characteristic properties as a multi-purpose plasmid vector.

1. Okayama-Berg method and Gubler-Hoffman method can be applied.

2. Plasmid yield per cultured cells is high.

3. Single-stranded DNA can be prepared.

4. A cDNA insert can be cut out easily.

5. Preparation of a deletion mutant for sequencing use can be made easily.

6. In vitro transcription can be made.

7. It has a promoter which can be expressed in mammalian cells.

### EXAMPLE 2

### Construction of Vector for Selective cDNA Library for signal peptide

cDNA encoding hTac (human IL-2 receptor α, used as reporter gene) which was removed signal sequence was inserted into pUCSR α ML2 (previous described) to construct a plasmid and the plasmid was named pUCSR α-hTac. cDNA was inserted into the downstream of SR a promoter and the upstream of hTac gene of this vector. Fusion protein will be expressed on the membrane, when cDNA is translated into protein which have signal sequence and form fusion protein with hTac.

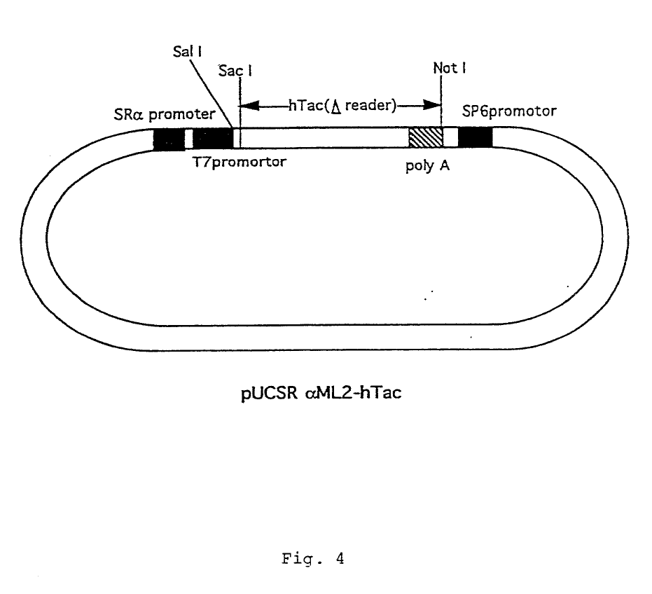

(1) That is, pBS-hTac which has a cDNA fragment encoded hTac inserted at hindIII site of pBluescript SK(+) (Stratagene, pBS) was digested at KpnI and the ends of the digested pBS-hTac were blunted by T4 DNA polymerase. Next, after the treated pBS-hTac was digested with Sacd to remove reader sequence, the resulting fragment was carried out SacI-EcoRI adapter ligation resulting in producing a fragment with a EcoRI end and a blunt end. The fragment was ligated with pUCSR α ML2 digested with EcoRI and SmaI, in which SacI site was broken to construct pUCSR α ML2-hTac (shown in FIG. 4.).

### EXAMPLE 3

### Preparation of cDNA Library Having Selectivity for Signal Peptide

Total RNA was extracted from ST2 cells, a mouse stromal cell line (that should support the survival and proliferation of hematopoietic stem cells and the proliferation and differentiation of B cells and myeloid cells; refer to EMBO J., 7, 1337 (1988))by the acid guanidine-phenol-chloroform (AGPC) method (described in detail in “Saibo Kogaku Jikken Protokoru (Protocol in Cellular Engineering Experiments)”, published by Shujun-sha, 28-31). Then poly A-RNA was purified by using oligo (dT)-latex (Oligotex-dT30, marketed from Takara Shuzo Co., Ltd.). By using a random hexamer as a primer, a single-stranded cDNA was synthesized with reverse transcriptase and dC was connected to the 3′-end thereof by terminal deoxytransferase. A 17 mer dC:

```
5′ GATGCGGCCGC GTCGAC GAATTC(dC)₁₇ 3′
          ------- ------ ------
            NotI SacI EcoRI
```

connected to some restriction enzyme site containing SalI was annealed with the fragment and a double-stranded cDNAs were synthesized by using the annealed oligo as a primer. Then the cDNA was fragmented by ultrasonication so as to give fragments in an average length of 500 bp and the fragmented cDNA in length of 400 to 800 bp were fractionated by agarose gel electrophoresis. After the ends of the separated fragments were blunted with T4 DNA polymerase, a lone linker:

```
5′ GAGGTACAAGCTT GATATC GAGCTCGCGG 3′
3′ CATGTTCGAA CTATAG CTCGAGCGCC 5′
                      ------ ------ ------
                  HindIII EcoRV SacI
```

containing an SacI site (see Nucleic Acids Res., 18, 4293(1990))was ligated and cDNAs of 400-800 bp were fractionated again by agarose gel electrophoresis. By using a primer (NLC):

5′GATGCGGCCGCGTCGACGAATTC3′

containing an SalI site and another primer (LLHES):

5′GAGGTACAAGCTTGATATCGAGCTCGCGG3′

containing an SacI site, PCR was performed 25 cycles at 94° C. for one minute, at 50° C. for two minutes and 72° C. for two minutes. The amplified cDNA was digested with SacI and EcoRI and cDNAs of 400-800 bp were fractionated by agarose gel electrophoresis.

The cDNA was ligated to a plasmid obtained by digesting pUCSR α ML2-hTac (prepared in example 2) with SacI and SalI by using T4 DNA ligase. After transformation of an DH5 α strain, a cDNA library having a selectivity for signal peptides was obtained.

### EXAMPLE 4

### Screening and Analysis of cDNA Coding for Signal Peptide

Colonies in the library obtained in Example 3 were divided into pools (ca 50 colonies/pool). Plasmids of each pools were isolated by the miniprep method and transfected in to COS-7 cells by the DEAD-dextran method (see Current Protocol in Molecular Biology,_@_{—}^{˜}9.2.1).

After 48 hours, cells were peeled from dish and were incubated with mouse Tac IgG antibody for 20 minutes on ice. After removal of free antibody, cells were incubated with goat anti-mouse IgG antibody labeled with FITC (fluorescent isothiocyanate) for 20 minutes on ice. Free antibody was removed, cells were performed fluorescent-staining for Tac of the cell surface.

Positive pools were selected under a fluorescent microscope. One positive pool were further divided colonies and the same procedure as described above was repeated until a single clone was obtained. Thus a positive single clone (ST-NT5) was obtained.

Subsequently, by using two synthetic primers:

5′GCCTGTACGGAAGTGTTACTTCTGC3′

(12 base upstream from PstI cloning site, for sense) and

5CCATGGCTTTGAATGTGGCG3′

(20 base downstream form SacI cloning site, for antisense)

which were specific for the pUSCST α ML2-hTac vector, the ST-NT5 insert was determined. DNA sequencing was performed by cycle-sequence method with fluorescent dye-terminator of ABI (Applied Biosystems Inc.) based on dideoxy terminator method of Sanger, F et al.

DNA sequencer (Model 373A) of ABI (Applied Biosystems Inc.) was used at reading of the sequence. Homology search was performed on DNA and amino acid, it is cleared that ST-TN5 encodes unknown protein.

### EXAMPLE 5

### Screening of Full Length cDNA and Determination of Nucleotide Sequence

Preparation of cDNA library was carried out by Super Script (Registered Trade Mark) Random System (BRL) using with mRNA derived from mouse stromal cell strain ST2. cDNAs were ligated to λgt22A (BRL) with SalI and NotI arm which had been treated by phosphatase.

In vitro packaging was performed followed by the protocol of in vitro Packaging Kit LAMDA INN (Japan Gene), transformed phage were infected to host Escherichia coli Y1090 (r-) (BRL).

Then, cDNA library containing 1,000,000 plaques were obtained. Next, plasmid containing ST-TN5 was digested at SalI and NotI and ST-TN5 fragments were fractionated with agarose gel electrophoresis. Screening of the library was performed with ST-TN5 cDNA fragment as probe, many positive clone were obtained.

Among the positive clones, a clone which has ca 1.8 kbp length was used. SalI-NotI fragment cut from λgt22A vector was subcloned into pUCSR α ML2, thus obtained plasmid which was thought that full length clone was contained and was named pBS-SDF5.

300 bp in length of the nucleotide sequence at 5′-termini of SDF5 cDNA was determined using with T7 primer. We firstly confirmed that the sequence corresponding to ST-TN5 used as the probe exists at the region of 5′-end of SDF5 cDNA which is derived from phage library.

### EXAMPLE 6

### Sequencing of Full Length cDNA and Determination of Open-reading Frame

Sequencing of full length cDNA was determined followed by the description of Molecular Cloning (Sambrook, J., Fritsch, E. F. and Maniatis, T. , Cold Spring Harbor Laboratory Press, in 1989) by using random sequencing method.

That is, cDNA inserts were isolated and purified from pBS-SDF5 by SalI-NotI digestion. Inserts were ligated, fragmented and the ends of the fragments were blunted by T4 DNA polymerase, DNA fragments having ca 400 bp length were recovered.

DNA fragments obtained were cloned into SmaI site of plasmid vector BLUESCRIPT II (Stratagene) and Escherichia coli was transformed with the plasmid. We picked up 20 colonies at random, plasmid DNA was prepared, and 20 plasmids (all of them have cDNA fragment of SDF5 as insert) were performed DNA sequencing.

Sequencing of DNA and reading of the sequence were performed by the same method as described in example 4.

Sequence data of SDF5 cDNA fragments were edited to continuous sequence by DNA sequence linking program of DNASIS to obtain nucleotide sequence shown in SEQ ID NO. 3. Open-frame was determined from the full length cDNA sequence data, and translated into amino acid sequence to obtain SEQ ID NO. 1.

1. Substantially purified form of the polypeptide comprising the amino-acid sequence shown in SEQ ID NO. 1, homologue thereof, fragment thereof or homologue of the fragment.

2. A polypeptide, according to claim 1, comprising the amino-acid sequence shown in SEQ ID NO. 1.

3. A DNA encoding the polypeptide according to claim 2.

4. A DNA, according to claim 3, comprising the nucleotide sequence shown in SEQ ID NO. 2 or a fragment DNA selectively hybridized to the DNA.

5. A DNA, according to claim 3, comprising the nucleotide sequence shown in SEQ ID NO. 3 or a fragment DNA selectively hybridized to the DNA.

6. A replication or expression vector carrying the DNA according to claim 3 to 5.

7. A host cell transformed with the replication or expression vector according to claim 6.

8. A method for producing the polypeptide according to claim 1 or 2 which comprises culturing a host cell according to claim 7 under a condition effective to express the polypeptide according to claim 1 or 2.

9. A monoclonal or polyclonal antibody against the polypeptide according to claim 1 or 2.

10. A pharmaceutical composition containing the polypeptide according to claim 1 or 2 or the antibody according to claim 9, in association with pharmaceutically acceptable diluent and/or carrier.
